(19) United States
(12) Patent Application Publication
(10) Pub. No.: US 2005/0070516 A1
(43) Pub. Date: Mar. 31, 2005

(54) As-needed administration of an androgenic agent to enhance female desire and responsiveness

(75) Inventors: Leland F. Wilson, Menlo Park, CA (US); Peter Y. Tam, Redwood City, CA (US)

Correspondence Address: REED INTELLECTUAL PROPERTY LAW GROUP, 800 MENLO AVENUE, SUITE 210, MENLO PARK, CA, 94025, US

(73) Assignee: VIVUS INC., Mountain View, CA (US)

(21) Appl. No.: 10/990,667

(22) Filed: Nov. 16, 2004

Related U.S. Application Data
(63) Continuation-in-part of application No. 09/919,472, filed on Jul. 27, 2001.
Application No. 09/919,472 is a continuation-in-part of application No. 09/539,484, filed on Mar. 30, 2000, now Pat. No. 6,306,841.
Application No. 09/539,484 is a continuation of application No. 09/181,316, filed on Oct. 27, 1998, now abandoned.
Application No. 09/181,316 is a continuation-in-part of application No. 08/959,064, filed on Oct. 28, 1997, now Pat. No. 5,877,216.
Application No. 09/181,316 is a continuation-in-part of application No. 08/959,057, filed on Oct. 28, 1997, now abandoned.

(51) Int. Cl. A61K 31/56; A61K 9/22

(52) U.S. Cl. 514/177; 424/468

(57) ABSTRACT

A method is provided for enhancing a female individual's sexual desire and responsiveness. The method involves administration of a pharmaceutical formulation containing an effective amount of an androgenic agent, wherein administration is on an as-needed basis rather than involving chronic pharmacotherapy. Local delivery may be accomplished via administration to the vagina, vulvar area or urethra of the individual, although oral administration is preferred for those androgenic agents that are orally active. Formulations and kits for carrying out the method are provided as well.

### TECHNICAL FIELD

This invention relates generally to methods and pharmaceutical formulations for enhancing female sexual desire and responsiveness, and more particularly, relates to the use of an androgenic agent in such methods and formulations.

### BACKGROUND

Sexual response in women is generally classified into four stages: excitement, plateau, orgasm, and resolution. Masters and Johnson, Human Sexual Response (Boston, Mass.: Little, Brown & Co., 1966). With sexual arousal and excitement, vasocongestion and muscular tension increase progressively, primarily in the genitals, and is manifested by increased blood flow, elevated luminal oxygen tension, and vaginal surface lubrication as a result of plasma transudation that saturates the fluid reabsorptive capacity of the vaginal epithelium. Sexual excitement is initiated by any of a number of psychogenic or somatogenic stimuli and must be reinforced to result in orgasm. With continued stimulation, excitement progresses in intensity into a plateau stage, from which the individual can shift into orgasm. The orgasmic stage is characterized by a rapid release from vasocongestion and muscular tension.

Disorders of female sexual desire or response are estimated to affect from 30 to 50 percent of the adult population in various studies (see, e.g., Nathon (1986), “The Epidemiology of the DSM-III Psychosexual Dysfunctions,” J. of Sex and Marital Therapy, 12(4):267-281; Diagnostic and Statistical Manual IV, “Sexual and Gender Identity Disorder,” American Psychiatric Association, Washington. D.C., pp.493-539, 1994; Osborn et al. (1988), “Sexual Dysfunction Among Middle Aged Women in the Community,” British Medical Journal 296:959-962; Frank et al. (1978), “Frequency of Sexual Dysfunction in ‘Normal Couples’,” New England Journal of Medicine, 299:111-115; and Garde et al. (1980), “Female Sexual Behavior: A Study in a Random Sample of Forty-year-old Danish Women,” Maturitas 2:225-240). Sexual dysfunction may be due to organic or functional disturbances. For example, a variety of diseases affecting neurologic function, including diabetes mellitus and multiple sclerosis, may interrupt sexual arousal. More commonly, local pelvic disorders, such as endometriosis and vaginitis, both of which cause dyspareunia (difficult or painful coitus), can also affect a woman□s sexual response. In addition, estrogen deficiency, causing vaginal atrophy and dyspareunia, is a common cause of sexual dysfunction. For a discussion of other causes of female sexual dysfunction, see, e.g., Kaplan, The Evaluation of Sexual Disorders: Psychological and Medical Aspects (New York: Brunner-Mazel, 1983), and Kolodny et al., Textbook of Sexual Medicine (Boston, Mass.: Little, Brown & Co., 1979).

Excitement stage dysfunction generally involves touch sensation impairment, loss of clitoral sensation, and vaginal dryness. Such excitement phase dysfunction generally results in dyspareunia. Dyspareunia is thought to affect approximately 40% of women, due in large part to inadequate lubrication. It has been estimated that over 40 million women will suffer dyspareunia at some time in their lives. On the order of twenty-five million will experience dyspareunia in the peri- and postmenopausal period (see Kelly, S. (1992) Clinical Practice and Sexuality 8(8):2 and Sato et al. (1992) Clinical Practices in Sexuality 8(5):1). Contemporary symptomatic treatments generally involve the use of physiologically safe lubricants such as egg white, K-Y surgical lubrication jelly (hydroxyethyl-cellulose), Astroglide□, and Replens□. See, for example, Semmens (1974) Medical Aspects of Human Sexuality 8:85-86, and Frishmen et al. (1992) Fertility and Sterility 58(3):630. When symptomatic treatment fails, pharmacological treatment may be indicated.

Estrogen therapy is commonly used in the pharmacological treatment of sexual dysfunction in women. Estrogen-based therapies are generally used to increase mucous production, provide vasodilatory effects, or to increase the general health of the vagina. Nadelson et al., eds., Treatment Interventions in Human Sexuality (New York: Plenum Press, 1983). In such treatments, estrogen is administered orally, parenterally (e.g., by injection), or topically. With oral administration, the estrogen concentration encountered by the liver is generally four- to five-fold greater than estrogen levels in peripheral blood (the “first pass effect”). This effect may lead to an undesirable increase in the production of certain coagulation factors and renin substrates by the liver. Parenterally administered estrogen avoids the first pass effect in the liver. However, all estrogen-based therapies are known to increase the risk of endometrial hyperplasia endometrial cancer and breast cancer in treated individuals.

Because of the increased risk of endometrial hyperplasia and endometrial cancer encountered with unopposed estrogen therapies, estrogen/progestogen combinations have been employed. However, common side effects from administration of such combinations include uterine bleeding and the continuation of menstrual periods.

Drug therapy, other than with female hormones, has been described for treating female sexual dysfunction. For example, U.S. Pat. No. 4,507,323 to Stern describes the use of the anxiolytic m-chloro-α-t-butylamino-propiophenone in the treatment of sexual dysfunction in both male and female individuals. Pharmaceutical formulations containing the agent are described, which are presented in discrete units, e.g., cachets, tablets, capsules, ampules and suppositories, for oral or rectal delivery of the agent.

Additionally, U.S. Pat. No. 4,521,421 to Foreman describes the treatment of sexual dysfunction in male and female individuals using the stereoisomers of octahydropyrimido[4,5-g]quinolines, centrally acting dopamine agonists. U.S. Pat. No. 5,190,967 to Riley describes the treatment of sexual disorders in male and female individuals using heterocyclic benzodioxinopyrrole compounds, which, like the drugs described in the aforementioned patents, are centrally acting agents.

U.S. Pat. No. 5,565,466 to Gioco et al., U.S. Pat. No. 5,731,339 to Lowrey, and U.S. Pat. No. 5,773,457 to Nahoum pertain to methods for modulating the human sexual response, with the Gioco et al. and Lowrey patents emphasizing the utility of phentolamine as an active agent.

There is nevertheless an ongoing need in the art for an effective method to treat female sexual disorders or dysfunction, or simply enhance female sexual desire and response in a normal woman, i.e., a woman not suffering from any sexual disorders or dysfunction. An ideal method would not require ongoing (“chronic”) drug therapy or use of active agents with numerous and/or serious side effects.

### SUMMARY OF THE INVENTION

Accordingly, the present invention is addressed to the aforementioned need in the art, and provides a novel way to enhance female sexual desire and responsiveness, wherein drug administration is on an “as-needed” basis rather than involving chronic pharmacotherapy, and is highly effective in the vast majority of women. The method is premised on the generally accepted principle that testosterone is the primary hormone responsible for sexual desire in women, and that elevated testosterone levels, typically occurring approximately midway through the menstrual cycle, correlate with increased sexual desire. The method, compositions and dosage forms of the invention not only enhance female sexual desire and responsiveness, but are also useful in improving the tissue health of the female genitalia and preventing vaginal atrophy, preventing pain during intercourse as a result of dyspareunia, and alleviating vaginal itching and dryness associated with dyspareunia and other conditions.

In order to carry out the method of the invention, a selected androgenic agent is administered to a female individual to enhance sexual desire and responsiveness, and/or to improve tissue health of the female genitalia, prevent vaginal atrophy, prevent pain during intercourse, and alleviate vaginal itching and dryness; the individual may or may not be suffering from a sexual disorder or dysfunction. The active agent may be administered orally, topically or transmucosally, transdermally, by inhalation, or using any other route of administration. Oral administration, because of its convenience, is preferred for those active agents that have sufficient oral bioavailability. For other active agents, the preferred mode of administration involves vaginal delivery and/or topical application to the clitoris and the surrounding vulvar region.

In another embodiment, pharmaceutical compositions, dosage forms and packaged kits are provided to carry out the aforementioned method. Pharmaceutical compositions and dosage forms contain a therapeutically effective amount of the active agent, or a therapeutically effective concentration of the active agent, i.e., a concentration that provides a therapeutically effective amount of active agent upon administration of a selected volume of composition. Packaged kits include a pharmaceutical composition or dosage form containing the active agent, a container housing the composition or dosage form during storage and prior to administration, and instructions, e.g., written instructions on a package insert or label, for carrying out as-needed drug administration in a therapeutically effective manner. The composition or dosage form may be any of those described herein, e.g., an oral dosage form containing a unit dosage of a selected androgenic agent, the unit dosage being a therapeutically effective dosage for enhancement of female sexual desire and responsiveness.

Additional objects, advantages and novel features of the invention will be set forth in part in the description which follows, and in part will become apparent to those skilled in the art upon examination of the following, or may be learned by practice of the invention.

### DETAILED DESCRIPTION OF THE INVENTION

I. Definitions and Nomenclature:

Before describing the present invention in detail, it is to be understood that this invention is not limited to specific dosage forms, carriers, or the like, as such may vary. It is also to be understood that the terminology used herein is for the purpose of describing particular embodiments only, and is not intended to be limiting.

It must be noted that as used in this specification and the appended claims, the singular forms “a,” “an” and “the” include plural referents unless the context clearly dictates otherwise. Thus, for example, reference to “an active agent” or “a pharmacologically active agent” includes a single active agent as well as two or more different active agents in combination, reference to “an androgenic agent” includes a single androgenic agent as well as combinations of different androgenic agents, reference to “a carrier” includes mixtures of two or more carriers as well as a single carrier, and the like. In describing and claiming the present invention, the following terminology will be used in accordance with the definitions set out below.

The terms “active agent,” “pharmacologically active agent” and “drug” are used interchangeably herein to refer to a chemical compound that induces a desired pharmacological, physiological effect, i.e., in this case, enhancement of female sexual desire and responsiveness. The primary active agents herein are androgenic agents. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when an active agent such as an androgenic agent is specifically identified, it is to be understood that applicants intend to include the active agent per se as well as pharmaceutically acceptable, pharmacologically active salts, esters, amides, prodrugs, metabolites, analogs, etc. The primary active agents herein are androgenic agents.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active “derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective to enhance female sexual desire and responsiveness.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

By an “effective” amount or a “therapeutically effective amount” of a drug or pharmacologically active agent is meant a nontoxic but sufficient amount of the drug or agent to provide the desired effect, i.e., enhancement of female sexual desire and responsiveness. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” sexual dysfunction, as the term is used herein, encompasses both prevention of sexual dysfunction in clinically asymptomatic individuals and treatment of dysfunction in a clinically symptomatic individual.

By “as-needed” dosing, also referred to as “pro re nata” dosing, “prm” dosing, and “on-demand” dosing or administration, is meant the administration of an active agent at a time just prior to the time at which drug efficacy is wanted, e.g., just prior to anticipated sexual activity, and within a time interval sufficient to provide for the desired therapeutic effect, i.e., enhancement in sexual desire and in sexual responsiveness during sexual activity. “As-needed” administration herein does not involve priming doses or chronic administration, “chronic” meaning administration at regular time intervals on an ongoing basis. As-needed administration may involve administration immediately prior to sexual activity, but will generally be about 0.25 to 72 hours, preferably about 0.5 to 48 hours, more preferably about 1 to 24 hours, most preferably about 1 to 12 hours, and optimally about 1 to 4 hours prior to anticipated sexual activity. “As-needed” administration may or may not involve administration of a sustained release formulation in advance of anticipated sexual activity, with drug release taking place throughout an extended drug delivery period typically in the range of about 4 to 72 hours.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). As discussed therein, immediate and nonimmediate release can be defined kinetically by reference to the following equation:

$$\begin{matrix}Dosage \\ Form\end{matrix} {{\to }^{{k}_{r}}}_{\begin{matrix}drug \\ release\end{matrix}} \begin{matrix}Absorption \\ Pool\end{matrix} {{\to }^{{k}_{a}}}_{absorption} \begin{matrix}Target \\ Area\end{matrix} {{\to }^{{k}_{e}}}_{elimination}$$

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area. The term “controlled release” as used herein includes any nonimmediate release formulation, including but not limited to sustained release, delayed release and pulsatile release formulations.

The term “sustained release” is used in its conventional sense to refer to a drug formulation that provides for gradual release of a drug over an extended period of time, and that preferably, although not necessarily, results in substantially constant blood levels of a drug over an extended time period. A sustained release formulation may be administered once to provide a single bolus dose of the androgenic agent, which is then effective for up to a day or even up to several days.

By the term “transdermal” drug delivery is meant delivery by passage of a drug through the skin or mucosal tissue and into the bloodstream. “Transdermal” delivery is also intended to encompass passage through scrotal skin.

The term “topical administration” is used in its conventional sense to mean delivery of a topical drug or pharmacologically active agent to the skin or mucosa. Topical administration thus includes transmucosal administration.

By “transmucosal” drug delivery or administration is meant administration of a drug to the mucosal surface of an individual so that the drug passes through the mucosal tissue and into the individual's blood stream. Transmucosal drug delivery may be “buccal” or “transbuccal,” referring to delivery of a drug by passage through an individual's buccal mucosa and into the bloodstream. Another form of transmucosal drug delivery herein is “sublingual” drug delivery, which refers to delivery of a drug by passage of a drug through an individual's sublingual mucosa and into the bloodstream. “Transmucosal delivery” also includes “vaginal delivery,” i.e., direct administration of a pharmaceutical formulation to the vagina of the individual undergoing treatment. Generally, “vaginal delivery” of a pharmaceutical formulation involves administration to the distal several centimeters of the vagina. The terms “vulvar delivery” and “vulvar administration” are used herein to refer to application of a pharmaceutical formulation to the vulvar area of an individual undergoing treatment. The term is intended to encompass application to the clitoris as well as the surrounding vulvar area. The terms “vulvar delivery” and “clitoral delivery” are used interchangeably herein and are both intended to refer to administration to the vulvar area of the individual undergoing treatment.

In order to carry out the method of the invention, a selected androgenic agent is administered on an as-needed basis to a female individual to enhance sexual desire and responsiveness; the individual may or may not be suffering from a sexual disorder or dysfunction. The active agent may be administered orally, topically or transmucosally (including buccally, sublingually, vaginally, etc., as well as administration to the vulvar region), transdermally, by inhalation, or using any other route of administration. Oral administration, because of its convenience, is preferred for those active agents that have sufficient oral bioavailability.

By “enhancing female sexual desire and responsiveness” applicants intend to include the treatment of disorders of female sexual desire and/or response, meaning any disorder or dysfunction that causes a decrease in or absence of female sexual responsiveness or female sexual desire. This includes any persistent or recurrent deficiency in the desire for sexual activity. It also includes decreases in the physiological response to sexual stimulation such as slowed or decreased erectile response of the female erectile tissues; slowed, decreased or absent lubrication of the vagina; slowed, decreased, or absent ability to have orgasms; decreased intensity of or pleasure in orgasms: frigidity; sexual aversion; and disorders of female sexual desire and response that are secondary to a general medical condition such as the menopausal or post-menopausal state, radiotherapy of the pelvis, atherosclerosis, pelvic trauma or surgery, peripheral neuropathies, autonomic neuropathies, diabetes mellitus, and disorders of the innervation of any of the sexual organs. This term also includes substance-induced sexual dysfunction, including but not limited to, decreases in desire and responsiveness secondary to anti-depressants, neuroleptics, anti-hypertensives, tobacco, opiates, alcohol and any other drug found to decrease or eliminate any part of the sexual response cycle. Primary and secondary anorgasmia are included.

II. Active Agents:

A. Androgenic Agents

The primary active agent herein is an androgenic agent. As will be discussed in further detail infra, the primary active agent may be administered alone or in conjunction with one or more secondary active agents. Suitable androgenic agents include, but are not limited to:
the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, dromostanolone propionate, testosterone, dehydroepiandrosterone (DHEA; also termed “prasterone”), sodium dehydroepiandrosterone sulfate, and 4-dihydrotestosterone (DHT; also referred to as “stanolone” and 5α-dihydrotestosterone);
pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, pentadecanoate, undecanoate, pelargonate, tridecanoate, palmitate, caprate, isocaprate, α-methylcaprate, β-methylcaprate, laurate, α-methylpelargonate, β-methylpelargonate, β,β-dimethylpelargonate, β-(p-methyl-cyclohexyl)propionate, β-(p-ethylcyclohexyl)-propionate, β-(cycloheptyl)-propionate, α-methyl-β-cyclohexyl-propionate, β-methyl-β-cyclohexyl-propionate, cyclododecyl-carboxylate, adamantine-1′-carboxylate, adamant-1′-yl-acetate, methyl-β-cyclohexyl propionate, and β-(bicyclo-[2,2,2-oct-1′-yl)-propionate esters, as well as the alkyl-substituted, preferably C₄-C₆ alkyl-substituted cyclic esters, such as the 3-n-hexylcyclo-butanecarboxylate, 3-n-butylcyclopentanecarboxylate, 4-n-butylcyclohexanecarboxylate, 4-n-pentylcyclohexanecarboxylate and n-hexylcyclohexanecarboxylate esters (and other such esters disclosed in U.S. Pat. No. 4,948,790); and
pharmaceutically acceptable derivatives of testosterone such as methyl testosterone, testolactone, oxymetholone, fluoxymesterone, and the like,
including combinations of any of the foregoing.

Those androgenic agents having suitable oral bioavailability may be advantageously administered orally. Orally active androgenic agents include, without limitation, testosterone propionate, undecanoate, and C₄-C₆ alkyl-substituted cycloalkylcarboxylates, as alluded to above, as well as the propionate, undecanoate, and C₄-C₆ alkyl-substituted cycloalkylcarboxylate esters of 4-dihydrotestosterone. Other androgenic agents that have oral activity, and whose oral activity can be enhanced by admixture with a lipoidal vehicle, include those mentioned in U.S. Pat. No. 4,147,783 to van der Vies, including, by way of example, the following esters of testosterone and DHT: decanoate, pentadecanoate, undecanoate, pelargonate, tridecanoate, palmitate, caprate, isocaprate, α-methylcaprate, β-methylcaprate, laurate, α-methylpelargonate, β-methylpelargonate, β,β-dimethylpelargonate, β-(p-methylcyclohexyl)propionate, β-(p-ethylcyclohexyl)-propionate, β-(cycloheptyl)-propionate, α-methyl-β-cyclohexyl propionate, β-methyl-β-cyclohexyl-propionate, cyclododecylcarboxylate, adamantine-1′-carboxylate, adamant-1′-yl-acetate, methyl-β-cyclohexyl-propionate, and β-(bicyclo-[2,2,2-oct-1′-yl)-propionate esters. Suitable lipoidal vehicles for enhancing the oral activity of the aforementioned esters are oils, e.g., arachis oil, castor oil, sesame oil, linseed oil, soya bean oil, sunflower seed oil, olive oil, fish liver oil, ethyl oleate, oleyl oleate, glyceryl trioleate, glyceryl dioleate, glyceryl monooleate, and oleic acid.

B. Secondary Active Agents

Additional pharmacologically active agents may be co-administered along with the primary active agent, i.e., with the androgenic agent. Such additional active agents are also referred to herein as “secondary” active agents. Preferred secondary agents are vasoactive agents, particularly vasodilators, selected from the group consisting of vasoactive prostaglandins, endothelin-derived relaxation factors, vasoactive intestinal polypeptide agonists, smooth muscle relaxants, leukotriene inhibitors, pharmaceutically acceptable salts, esters, analogs, derivatives, prodrugs, active metabolites, and inclusion complexes thereof, and combinations of any of the foregoing. Other suitable secondary agents include rho kinase inhibitors, melanocortin peptides, endothelin antagonists, growth factors and other peptidyl drugs; selective androgen receptor modulators (SARMs), neuropeptides, amino acids, serotonin agonists, serotonin antagonists, calcium channel blockers, potassium channel openers, potassium channel blockers, dopamine agonists, dopamine antagonists, non-androgenic steroid hormones, and combinations thereof.

Particularly preferred vasoactive agents are vasoactive prostaglandins selected from the group consisting of naturally occurring prostaglandins, semisynthetic prostaglandins, synthetic prostaglandins, and pharmaceutically acceptable, pharmacologically active salts, esters, amides, inclusion complexes prodrugs, metabolites, and analogs thereof. Racemic, optically enriched or purified stereoisomers of any of these compounds are also included. A suitable unit dose of a prostaglandin herein is in the range of approximately 1 to 5000 μg, preferably in the range of approximately 20 to 2000 μg. Preferred prostaglandins include, but are not limited to, the naturally occurring prostaglandins prostaglandin E₀ (PGE₀, also referred to 13,14-dihydro-PGE₁; hereinafter, the abbreviation “PG” is used for “prostaglandin”), PGE₁, 19-hydroxy-PGE₁, PGE₂, 19-hydroxy-PGE₂, PGA₁, 19-hydroxy-PGA₁, PGA₂, 19-hydroxy-PGA₂, PGB₁, 19-hydroxy-PGB₁, PGB₂, 19-hydroxy-PGB₂, PGB₃, PGD₂, PGF_{1α}, PGF_{2α}(dinoprost), PGE₃, PGF_{3α}, PGI₂ (prostacyclin), and combinations thereof. PGE₀, PGE₁, PGE₂, and the hydrolyzable lower alkyl esters thereof (e.g., the methyl, ethyl and isopropyl esters) are, however, particularly preferred. Other suitable prostaglandins are exemplified, without limitation, by arboprostil, carbaprostacyclin, carboprost tromethamine, dinoprost tromethamine, dinoprostone, enprostil, iloprost, lipoprost, gemeprost, metenoprost, sulprostone, tiaprost, viprostil (CL 115,347), viprostil methyl ester, 16,16-dimethyl-Δ²-PGE₁ methyl ester, 15-deoxy-16-hydroxy-16-methyl-PGE₁ methyl ester (misoprostol), 16,16-dimethyl-PGE₁, 11-deoxy-15-methyl-PGE₁, 16-methyl-18,18,19,19-tetrahydrocarbacyclin, 16(RS)-15-deoxy-16-hydroxy-16-methyl-PGE₁ methyl ester, (+)-4,5-didehydro-16-phenoxy-α-tetranor-PGE₂ methyl ester, 11-deoxy-11α,16,16-trimethyl-PGE₂, (+)-11α,16α,16β-dihydroxy-1,9-dioxo-1-(hydroxymethyl)-16-methyl-trans-prostene, 9-chloro-16,16-dimethyl-PGE₂, 16,16-dimethyl-PGE₂, 15(S)-15-methyl-PGE₂, 9-deoxy-9-methylene-16,16-dimethyl-PGE₂, potassium salt, 19(R)-hydroxy-PGE₂, and 11-deoxy-16,16-dimethyl-PGE₂.

Additional vasoactive agents useful as secondary active agents herein include endothelin-derived relaxation factors (“EDRFs”) such as nitric oxide releasing agents, e.g., sodium nitroprusside and diazenium diolates, or “NONOates.” NONOates include, but are not limited to, (Z)-1-{N-methyl-N-[6-(N-methyl-ammoniohexyl)amino]}diazen-1-ium-1,2-diolate (“MAHMA/NO”), (Z)-1-[N-(3-ammoniopropyl)-N-(n-propyl)amino]-diazen-1-ium-1,2-diolate (“PAPA/NO”), (Z)-1-{N-[3-aminopropyl]-N-[4-(3-aminopropylammonio)butyl]amino}diazen-1-ium-1,2-diolate (spermine NONOate or “SPER-NO”) and sodium (Z)-1-(N,N-diethylamino)-diazen-1-ium-1,2-diolate (diethylamine NONOate or “DEA/NO”) and derivatives thereof). Still other vasoactive agents include vasoactive intestinal polypeptide analogs and derivatives thereof (particularly derivatives in the form of hydrolyzable lower alkyl esters), smooth muscle relaxants, leukotriene inhibitors, calcium channel blockers, β2-adrenergic agonists, angiotensin-converting enzyme (“ACE”) inhibitors, angiotensin II receptor antagonists, and phosphodiesterase inhibitors.

Still other suitable vasoactive agents include, but are not limited to: nitrates and like compounds such as nitroglycerin, isosorbide dinitrate, erythrityl tetranitrate, amyl nitrate, molsidomine, linsidomine chlorhydrate (“SIN-1”), S-nitroso-N-acetyl-d,1-penicillamine (“SNAP”) and S-nitroso-N-glutathione (“SNO-GLU”); long and short acting α-blockers such as phenoxybenzamine, dibenamine, doxazosin, terazosin, phentolamine, tolazoline, prazosin, trimazosin, alfuzosin, tamsulosin and indoramin; ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; nimodepine; pinacidil; cyclandelate; dipyridamole; isoxsuprine; chlorpromazine; haloperidol; yohimbine; and trazodone.

Other secondary active agents herein are inhibitors of rho kinase, an enzyme belonging to the rhoA/rho associated kinase pathway, which regulates the state of phosphorylation of myosin phosphatase, in turn leading to the control of smooth muscle contraction. One example of a suitable rho kinase inhibitor has the structural formula 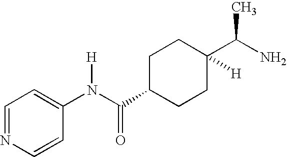
and is identified as Y-27632. Other suitable rho kinase inhibitors are disclosed, for example, in U.S. Pat. No. 6,218,410.

Additional secondary agents useful herein are peptide analogs of α-melanocyte-stimulating hormone (α-MSH), also referred to as “melanocortin peptides.” Such peptides include the sequence His-Phe-Arg-Trp, His-D-Phe-Arg-Trp, or are homologs thereof, and are preferably cyclic. A preferred melanocortin peptide is Ac-Nle-cyclo(-Asp-His-D-Phe-Arg-Trp-Lys)-OH. See U.S. Pat. No. 6,051,555 to Hadley and International Patent Publication No. WO 01/00224 to Blood et al., assigned to Palatin Technologies, Inc. The aforementioned amino acid residues have their conventional meaning as given in Chapter 2422 of the Manual of Patent Examining Procedure (2000). Thus, “Arg” is arginine, “Nle” is norleucine, “His” is histamine, “Phe” is phenylalanine, “D-Phe” is D-phenylalanine, “Trp” is tryptophan, and “Ac” refers to an acetyl moiety, i.e., an acetyl moiety present in a peptide or amino acid sequence that is acetylated.

Suitable endothelin antagonists include antagonists of any or all of the three isoforms of endothelin, i.e., ET-1, ET-2, and ET-3, and are exemplified by: phenoxyphenylacetic acids and derivatives thereof, such as N-(4-isopropylbenzene-sulfonyl)-α-(4-carboxy-2-n-propylphenoxy)-3,4-methylenedioxyphenyl acetamide dipotassium salt, 2-[(2,6-dipropyl-4-hydroxymethyl)-phenoxy]-2-(4-phenoxyphenyl)-acetic acid, 2-[(2,6-dipropyl-4-hydroxymethyl)phenoxy]-2-(4-phenylphenyl)acetic acid, 2-[(2,6-dipropyl-4-hydroxymethyl)phenoxy]-2-(3-carboxyphenyl)-acetic acid, 2-[(2,6-dipropyl-4-hydroxymethyl)phenoxy]-2-(3,4-ethylenedioxyphenyl)acetic acid, 2-[(2,6-dipropyl-4-hydroxymethyl)phenoxy]-2-(3,4,5-trimethoxyphenyl)acetic acid, 2-[(2,6-dipropyl-4-hydroxymethyl)phenoxy]-2-(3,4-methylenedioxyphenyl)acetic acid, N-(4-dimethylaminobenzenesulfonyl)-2-(4-methoxycarbonyl-2-propylphenoxy)-2-(3,4-methylenedioxyphenyl)acetamide, N-(2-methylbenzenesulfonyl)-2-(4-methoxycarbonyl-2-propylphenoxy)-2-(3,4-methylenedioxyphenyl)acetamide, N-(2-methoxycarbonyl-benzenesulfonyl)-2-(4-methoxy-carbonyl-2-propylphenoxy)-2-(3,4-methylenedioxy-phenyl)acetamide, N-(2-chlorobenzene-sulfonyl)-2-(4-methoxycarbonyl-2-propylphenoxy)-2-(3,4-methylenedioxyphenyl)acetamide, and others, as described in U.S. Pat. No. 5,565,485; and certain isooxazoles, oxazoles, thiazoles, isothiazoles and imidazoles, as described, for example, in U.S. Pat. No. 6,136,828. Numerous other endothelin antagonists may be used as secondary agents herein, and will be known to those of ordinary skill in the art and/or are described in the pertinent patents, literature and texts.

Peptidyl drugs include the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin.

Selective androgen receptor modulators (SARMs) include LGD2226 and/or LGD1331, both available from Ligand Pharmaceuticals (San Diego, Calif.). See Negro-Villar et al. (1999) J. Clin. Endocrinol. & Metabol. 84(10):3459-62.

Suitable neuropeptides include bradykinin, kallidin, des-Arg⁹-bradykinin, des-Arg¹⁰-kallidin, des-Arg⁹-[Leu⁸]-bradykinin, [D-Phe⁷]-bradykinin, HOE 140, neuropeptide Y, calcitonin gene-related peptide (cGRP), enkaphalins and related opioid peptides such as Met⁵-enkaphalin, Leu⁵-enkephalin, α-, β- and γ-endorphin, α- and β-neo-endorphin, and dynorphin, as well as the neurotransmitters GABA (γ-aminobutyric acid), glycine, glutamate, acetylcholine, dopamine, epinephrine, 5-hydroxytryptamine, substance P, serotonin, and catecholamines.

One or more amino acids may be included in the present formulations. As used herein, the term “amino acid” includes the conventional amino acids, e.g., phenylalanine, leucine, isoleucine, methionine, valine, serine, proline, threonine, alanine, tyrosine, histidine, glutamine, asparagine, lysine, aspartic acid, glutamic acid, cysteine, tryptophan, arginine, and glycine, with arginine being particularly preferred. In addition, the term “amino acid” will also include amino acid derivatives, e.g., 1-naphthylalanine, 2-naphthylalanine, 3-pyridylalanine, 4-hydroxyproline, O-phosphoserine, N-acetylserine, N-formylmethionine, 3-methylhistidine, 5-hydroxylysine, and nor-leucine, in addition to stereoisomers (e.g., D-amino acids) of the twenty conventional amino acids. Combinations of any of the foregoing are contemplated as well. Preferred amino acids are the neuroactive amino acids γ-aminobutyric acid (GABA), glycine, β-alanine, taurine, and glutamate.

Suitable serotonin agonists include, but are not limited to 2-methyl serotonin, buspirone, ipsaperone, tiaspirone, gepirone, ergot alkaloids, 8-hydroxy-(2-N,N-dipropyl-amino)-tetraline, 1-(4-bromo-2,5-dimethoxyphenyl)-2-aminopropane, cisapride, sumatriptan, m-chlorophenylpiperazine, trazodone, zacopride, mezacopride, and combinations thereof. Suitable serotonin antagonists include, for example, ondansetron, granisetron, metoclopramide, tropisetron, dolasetron, palonosetron, trimethobenzamide, methysergide, risperidone, ketanserin, ritanserin, clozapine, amitriptyline, MDL 100,907 (R(+)-α-(2,3-dimethoxyphenyl)-1-[2-(4-fluorophenyl)ethyl]-4-piperidine-methanol) (Marion Merrell Dow), azatadine, cyproheptadine, fenclonine, chlorpromazine, mianserin and combinations thereof.

Representative ergot alkaloids include ergotamine and ergotamine analogs, e.g., acetergamine, brazergoline, bromerguride, cianergoline, delorgotrile, dihydroergotamine, disulergine, ergonovine, ergonovine maleate, ergotamine tartrate, etisulergine, lergotrile, lysergide, mesulergine, metergoline, metergotamine, nicergoline, pergolide, propisergide, proterguride and terguride.

Calcium channel blockers that are suitable for use according to the present invention include, without limitation, amlodipine, felodipine, isradipine, nicardipine, nifedipine, nimodipine, nisoldipine, nitrendipine, bepridil, diltiazem, verapamil, and combinations thereof.

Potassium channel openers include, but are not limited to, pinacidil, diazoxide, cromakalim, nicorandil, minoxidil, (N-cyano-N′-(1,1-dimethylpropyl)-N″-3-pyridyl-guanidine (P-1075), and N-cyano-N′-(2-nitroxyethyl)-3-pridinecarboximidamide monomethanesulfonate (KRN 2391).

Potassium channel blockers include tedisamil, agitoxin-2, apamin, BDS-I, BDS-II, charybdotoxin, α-dendrotoxin, β-dendrotoxin, γ-dendrotoxin, δ-dendrotoxin, dendrotoxin-I, dendrotoxin-K, E-4031, iberiotoxin, kaliotoxin, MCD-peptide, margatoxin, noxiustoxin, paxilline, penitrem A, stichodactyla, tertiapin, tityustoxin K alpha, verruculogen, and combinations thereof. Although all of the active agents are available commercially, most of the listed potassium channel blockers are available from Alomone Labs (Jerusalem, Israel).

Suitable dopamine agonists include, for example, levodopa, bromocriptine, pergolide, apomorphine, piribedil, pramipexole, ropinirole, and combinations thereof. Dopamine antagonists include, without limitation, spiroperidol, benperidol, trifluperidol, pimozide, fluphenazine, droperidol, haloperidol, thiothixene, trifluperazine, moperone, prochlorperazine, molindone, thioridazine, clozapine, chlorpromazine, promazine, sulpiride, clebopride, chlorpromazine, spiperone, flupenthixol, and combinations thereof.

Non-androgenic steroids that may be administered as secondary active agents include progestins and estrogens. Suitable estrogens include synthetic and natural estrogens such as: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Suitable progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. It is generally desirable to co-administer a progestin along with an estrogen so that the estrogen is not “unopposed.” As is well known in the art, estrogen-based therapies are known to increase the risk of endometrial hyperplasia and cancer, as well as the risk of breast cancer, in treated individuals. Co-administration of estrogenic agents with a progestin has been found to decrease the aforementioned risks.

The androgenic agent and the additional active agent or agents may be incorporated into a single formulation, or they may be administered separately, either simultaneously or sequentially. In a preferred embodiment, the androgenic agent is administered prior to administration of a vasoactive agent such as a prostaglandin, i.e., the androgenic agent is administered as a pretreatment. In a particularly preferred embodiment, such a method involves administration of an androgenic agent, e.g., via oral or topical (preferably vulvar and/or vaginal) administration, followed by topical (again, preferably vulvar and/or vaginal) administration of a topical prostaglandin formulation as described in U.S. Pat. No. 5,877,216, preferably a topical formulation containing prostaglandin E₀, prostaglandin E₁, or prostaglandin E₂, most preferably prostaglandin E₁.

C. Derivatives

Any of the active agents may be administered in the form of a salt, ester, amide, prodrug, active metabolite, analog, or the like, provided that the salt, ester, amide, prodrug, active metabolite or analog is pharmaceutically acceptable and pharmacologically active in the present context. Salts, esters, amides, prodrugs, metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

For example, acid addition salts are prepared from the free base using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of acid moieties which may be present on an active agent may be carried out in a similar manner using a pharmaceutically acceptable base such as sodium hydroxide, potassium hydroxide, ammonium hydroxide, calcium hydroxide, trimethylamine, or the like. Preparation of esters involves transformation of a carboxylic acid group via a conventional esterification reaction involving nucleophilic attack of an RO⁻ moiety at the carbonyl carbon. Esterification may also be carried out by reaction of a hydroxyl group with an esterification reagent such as an acid chloride. Esters of testosterone and other androgenic agents having a 17β-hydroxyl group are usually formed at that hydroxyl group, i.e., are 17β-esters. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs and active metabolites may also be prepared using techniques known to those skilled in the art or described in the pertinent literature. Prodrugs are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature. In addition, chiral active agents may be in isomerically pure form, or they may be administered as a racemic mixture of isomers.

III. Pharmaceutical Compositions and Dosage Forms:

Suitable compositions and dosage forms include tablets, capsules, caplets, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, and the like.

A. Oral Dosage Forms:

Oral dosage forms are preferred for those therapeutic agents that are orally active, and include tablets, capsules, caplets, solutions, suspensions and/or syrups, and may also comprise a plurality of granules, beads, powders or pellets that may or may not be encapsulated. Such dosage forms are prepared using conventional methods known to those in the field of pharmaceutical formulation and described in the pertinent texts, e.g., in Remington: The Science and Practice of Pharmacy, 20^{th} Edition, Gennaro, A. R., Ed. (Lippincott, Williams and Wilkins, 2000). Tablets and capsules represent the most convenient oral dosage forms, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), then, tablets prepared for oral administration using the method of the invention will generally contain other materials such as binders, diluents, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, magnesium stearate, calcium stearate, and stearic acid. Stearates, if present, preferably represent at no more than approximately 2 wt. % of the drug-containing core. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts, e.g., in Remington, supra. Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

The amount of androgenic agent per oral dosage unit, for example a tablet or capsule, may vary significantly, for example from 1 μg to about 250 mg, preferably from 1 μg to about 150 mg, most preferably in the range of about 10 μg to about 100 mg. Generally, although not necessarily, the unit dosage for oral administration will be somewhat to substantially higher than the unit dosages appropriate for other modes of administration.

B. Compositions and Dosage Forms for Administration to the Vulvar Region and/or Vagina:

With androgenic agents that are not orally active, the preferred mode of administration involves topical delivery to the vulvar region and/or vaginal drug administration. These pharmaceutical formulations will typically contain one or more pharmaceutically acceptable carriers suited to the particular type of formulation, i.e., gel, ointment, suppository, or the like. The vehicles are comprised of materials of naturally occurring or synthetic origin that do not adversely affect the active agent or other components of the formulation. Suitable carriers for use herein include water, silicone, waxes, petroleum jelly, polyethylene glycol, propylene glycol, liposomes, sugars such as mannitol and lactose, and a variety of other materials, again depending, on the specific type of formulation used. As described in Section IV, infra, dosage forms used for administration to the vulvar region and/or vagina may be used to deliver drug on an as-needed, on-demand basis, and/or throughout an extended, sustained release profile.

The formulations may also include a chemical compound to enhance permeation of the active agent through the mucosal tissue, i.e., a “permeation enhancer.” Suitable permeation enhancers include those generally useful in conjunction with topical, transdermal or transmucosal drug delivery. Examples of suitable permeation enhancers include the following: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclaza-cycloheptan-2-one (available under the trademark Azone® from Nelson Research & Development Co., Irvine, Calif.; see U.S. Pat. Nos. 3,989,816, 4,316,893, 4,405,616 and 4,557,934); alcohols such as ethanol, propanol, octanol, decanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid and succinic acid. Mixtures of two or more enhancers may also be used.

In some cases, the formulations may include an enzyme inhibitor, i.e., a compound effective to inhibit enzymes present in the vagina or vulvar area that could degrade or metabolize the active agent. That is, inhibitors of enzymes that decrease or eliminate the activity of the active agent may be included in the formulation so as to effectively inhibit the action of those enzymes. Such compounds include, for example, fatty acids, fatty acid esters, and NAD inhibitors.

The formulations may be in the form of an ointment, cream, emulsion, lotion, gel, solid, solution, suspension, foam or liposomal formulation. Alternatively, the formulations may be contained within a vaginal ring (e.g., as disclosed in U.S. Pat. No. 5,188,835 to Lindskog et al., assigned to Kabi Pharmacia AB), or within a tampon, suppository, sponge, pillow, puff, or osmotic pump system; these platforms are useful solely for vaginal delivery.

Ointments are semisolid preparations that are typically based on petrolatum or other petroleum derivatives. The specific ointment base to be used, as will be appreciated by those skilled in the art, is one that will provide for optimum drug delivery. As with other carriers or vehicles, an ointment base should be inert, stable, nonirritating and nonsensitizing. As explained in Remington: The Science and Practice of Pharmacy, supra, at pages 1034-1038, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy for further information.

Lotions are preparations that may be applied without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. It is generally necessary that the insoluble matter in a lotion be finely divided. Lotions will typically contain suspending agents to produce better dispersions as well as compounds useful for localizing the active agent in contact with the skin, e.g., methylcellulose, sodium carboxymethylcellulose, or the like.

Pharmaceutical emulsion formulations are generally formed from a dispersed phase (e.g., a pharmacologically active agent), a dispersion medium and an emulsifying agent. If desired, emulsion stabilizers can be included in the formulation as well. A number of pharmaceutically useful emulsions are known in the art, including oil-in-water (o/w) formulations, water-in-oil (w/o) formulations and multiple emulsions such as w/o/w or o/w/o formulations. Emulsifying agents suitable for use in such formulations include, but are not limited to, TWEEN 60®, Span 80®, cetostearyl alcohol, myristyl alcohol, glyceryl monostearate and sodium lauryl sulfate.

Pharmaceutical creams are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

The above pharmaceutical formulations are formed by dispersing the finely divided or dissolved active agent uniformly throughout the vehicle or base using conventional techniques, typically by a levigating the agent with a small quantity of the base to form a concentrate, which is then diluted geometrically with further base. Alternatively, a mechanical mixer may be used. Creams, lotions and emulsions are formed by way of a two-phase heat system, wherein oil-phase ingredients are combined under heat to provide a liquified, uniform system. The aqueous-phase ingredients are separately combined using heat. The oil and aqueous phases are then added together with constant agitation and allowed to cool. At this point, concentrated agents may be added as a slurry. Volatile or aromatic materials can be added after the emulsion has sufficiently cooled. Preparation of such pharmaceutical formulations is within the general skill of the art.

The active agent can also be incorporated into a gel formulation using known techniques. Two-phase gel systems generally comprise a suspension or network of small, discrete particles interpenetrated by a liquid to provide a dispersed phase and a liquid phase. Single-phase gel systems are formed by distributing organic macromolecules uniformly throughout a liquid such that there are no apparent boundaries between the dispersed and liquid phases. Suitable gelling agents for use herein include synthetic macromolecules (e.g., Carbomers®, polyvinyl alcohols and polyoxyethylene-polyoxypropylene copolymers), gums such as tragacanth, as well as sodium alginate, gelatin, methylcellulose, sodium carboxymethylcellulose, methylhydroxyethyl cellulose and hydroxyethyl cellulose. In order to prepare a uniform gel, dispersing agents such as alcohol or glycerin can be added, or the gelling agent can be dispersed by trituration, mechanical mixing or stirring, or combinations thereof.

Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylammonium (“DOTMA”) liposomes are available under the tradename Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (“DOPC”), dioleoylphosphatidyl glycerol (“DOPG”), dioleoylphoshatidyl ethanolamine (“DOPE”), among others. These materials can also be mixed with DOTMA in appropriate ratios. Methods for making liposomes using these materials are well known in the art.

Vaginal suppositories are typically manufactured with polyethylene glycol (PEG), polyethylene oxide and/or other low melting point or water-soluble polymers including fatty acid esters. Suppositories may also be applied to the vulvar region, in which case these dosage forms, which are solid at ambient temperature, rapidly melts when placed on the clitoris and within the vulvar region.

Typically, compositions and dosage forms for vulvar and/or vaginal administration will contain the androgenic agent in a concentration such that an effective amount of the agent is delivered with a single application of the composition. For example, in the case of a gel, ointment or cream, the composition will contain sufficient androgenic agent such that an effective amount of the agent is delivered by application of about 0.1 g to 1.0 g of the composition. Thus, for delivering about 1.0 μg to 100 mg, preferably about 0.05 mg to 50 mg, most preferably about 1.0 mg to 25 mg, of the androgenic agent, the gel, ointment or cream formulation will contain the androgenic agent at a concentration in the range of about 1.0 μg/g to 1.0 g/g, preferably 50 μg/g to 500 mg/g, most preferably 1.0 mg/g to 250 mg/g.

With vaginal suppositories, a total suppository weight in the range of about 0.1 g to 0.5 g is common. Thus, pharmaceutical compositions according to the present invention that are in the form of a suppository will contain the androgenic agent at a concentration of about 2.0 μg/g to 1.0 mg/g, preferably 100 μg/g to 500 mg/g, most preferably 2.0 mg/g to 250 mg/g. Since drug dosages typically vary from person to person, repeated applications may be used to achieve the desired effect.

Delivery of an “as-needed” or “on-demand” dose with topical formulations intended for application to the vulvar region, and/or with vaginal suppositories, is effected by using the appropriate carrier and, when necessary, excipients, for the particular active agent. For example, the active agent's affinity to the carrier must be lower than its affinity to the treated body surface. Optimally, the agent will have a relatively high affinity for the mucosal surface to which it is applied, and a relatively low affinity for the particular carrier. As the affinity of the agent for the body surface remains constant (assuming the agent does not change, e.g., hydrolyze, etc., upon contact with the body surface), a suitable carrier for use in the formulations described herein can be determined by routinely testing a series of different carriers containing the active agent and selecting those carriers that provide the greatest flux of the active agent to the intended tissue, e.g., clitoral tissue. Additionally, one or more permeation enhancers and/or detergents may also be present in the formulation to ensure a rate of delivery sufficient for on-demand administration. A combination of these approaches as well as other approaches may be used to effect delivery of an on-demand dose.

Once the initial, on-demand dose is delivered, the drug delivery system, if present, and/or any remaining formulation may be removed or may remain in place, depending on the preferences of the individual. Alternatively, the formulation and optional drug delivery system may be designed to provide both initial “on-demand” release of the active agent, i.e., as a single, bolus dose, as well as sustained release thereafter, e.g., pulsatile, continuous or cyclical drug release. Such systems can include, for example, osmotic release systems that are capable of delivering an initial, on-demand release of the active agent in addition to variable amounts of the agent in a pulsatile manner thereafter.

Other drug delivery platforms capable of providing an initial release of drug followed by a pulsatile, continuous or cyclical agent release profile include those formed from bioerodible polymers, wherein the active agent is dispersed within a polymer matrix that is surrounded by an immediate release coating of the agent. The immediate release coating ensures effective on-demand administration. The polymers forming the matrix are selected such that they bioerode in the presence of moisture, and provide for sustained agent release at readily predictable rates.

More particularly, release of the active agent can be controlled by dissolution (bioerosion) of a polymer using either encapsulated dissolution control or matrix dissolution control. In encapsulated dissolution control, the on-demand dose of the active agent is located within an outer polymeric or wax membrane that dissolves to provide an initial release of the agent. When the encapsulating membrane comprising an initial release of the agent has dissolved, a core containing additional active agent is then available for release and adsorption across the epithelial or mucosal surfaces of the vagina or vulvar area. Bioerodible coating materials may be selected from a variety of natural and synthetic polymers, depending on the agent to be coated and the desired release characteristics. Exemplary coating materials include gelatins, carnauba wax, shellacs, ethylcellulose, cellulose acetate phthalate or cellulose acetate butyrate. Following the immediate release of the agent, a uniform sustained release of the agent can be attained by compressing a population of particles of the agent with varying membrane thickness (e.g., varying erosion times) into a tablet form for a single administration.

In matrix dissolution control, the active agent is dissolved or dispersed within a matrix of, for example, an erodible wax. The agent is released for adsorption across the epithelial or mucosal surfaces of the vagina or vulvar area as the matrix bioerodes. The rate of agent availability is generally controlled by the rate of penetration of the dissolution media (i.e., vaginal fluids) into the matrix, wherein the rate of penetration is dependent on the porosity of the matrix material. Bioerodible matrix dissolution delivery systems can be prepared by compressing the active agent with a slowly soluble polymer carrier into a tablet or suppository form. There are several methods of preparing drug/wax particles including congealing and aqueous dispersion techniques. In congealing methods, the active agent is combined with a wax material and either spray-congealed, or congealed and then screened. For an aqueous dispersion, the active agent/wax combination is sprayed or placed in water and the resulting particles collected. Matrix dosage formulations can be formed by compaction or compression of a mixture of active agent, polymer and excipients. Of course, the active agent will also be located in an external coating of the matrix formulation to provide for immediate release of the active agent necessary for on-demand administration.

In an alternative embodiment, the pharmaceutical formulation is administered in the form of biodegradable adhesive film or sheet that adheres to the vulvar area. Such drug delivery systems are generally composed of a biodegradable adhesive polymer based on a polyurethane, a poly(lactic acid), a poly(glycolic acid), a poly(ortho ester), a polyanhydride, a polyphosphazene, or a mixture or copolymer thereof. Preferred biodegradable adhesive polymers include polyurethanes and block copolyurethanes containing peptide linkages, simple mixtures of polyurethanes and polylactides, and copolymers of acrylates and mono- or disaccharide residues.

C. Other Transmucosal Compositions and Dosage Forms:

Although the present compositions will generally be administered orally and “locally,” i.e., to the vagina and/or vulvar region, other modes of administration are suitable as well. For example, other modes of transmucosal administration may be advantageously employed. That is, the selected active agent may be administered to the buccal mucosa in an adhesive tablet or patch, sublingually administered by placing a solid dosage form under the tongue, administered nasally as droplets or a nasal spray, administered by inhalation of an aerosol formulation, a non-aerosol liquid formulation, or a dry powder, or placed within or near the rectum (“transrectal” formulations).

Preferred buccal dosage forms will typically comprise a therapeutically effective amount of the selected androgenic agent and a bioerodible (hydrolyzable) polymeric carrier that may also serve to adhere the dosage form to the buccal mucosa. The buccal dosage unit is fabricated so as to erode gradually over a predetermined time period, wherein drug delivery is provided essentially throughout. The time period is typically in the range of approximately 0.5 hours to 24 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. The “therapeutically effective amount” of androgenic agent in the dosage unit will of course depend on the potency of the agent and the intended dosage, which, in turn, is dependent on the particular individual undergoing treatment, the specific indication, and the like. The dosage unit will generally contain from approximately 1.0 wt. % to about 60 wt. % active agent, preferably on the order of 1 wt. % to about 30 wt. % active agent. With regard to the bioerodible (hydrolyzable) polymeric carrier, it will be appreciated that virtually any such carrier can be used, so long as the desired drug release profile is not compromised, and the carrier is compatible with the androgenic agent to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol”, which may be obtained from B.F. Goodrich, is one such polymer). Other suitable polymers include, but are not limited to: hydrolyzed polyvinylalcohol; polyethylene oxides (e.g., Sentry Polyox” water soluble resins, available from Union Carbide); polyacrylates (e.g., Gantrez”, which may be obtained from GAF); vinyl polymers and copolymers; polyvinylpyrrolidone; dextran; guar gum; pectins; starches; and cellulosic polymers such as hydroxypropyl methylcellulose, (e.g., Methocel”, which may be obtained from the Dow Chemical Company), hydroxypropyl cellulose (e.g., Klucel”, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone” XL, which may be obtained from GAF), cross-linked carboxylic methylcelluloses, such as croscarmelose (e.g., Ac-di-sol”, which may be obtained from FMC), alginic acid, and sodium carboxymethyl starches (e.g., Explotab”, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab”, which may be obtained from Stauffer), sugars that have been processed by cocrystallization with dextrin (e.g., co-crystallized sucrose and dextrin such as Di-Pak”, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Preferred sublingual dosage forms include sublingual tablets, creams, ointments and pastes. The tablet, cream, ointment or paste for sublingual delivery comprises a therapeutically effective amount of the selected androgenic agent and one or more conventional nontoxic carriers suitable for sublingual drug administration. The sublingual dosage forms of the present invention can be manufactured using conventional processes. The sublingual dosage unit is fabricated to disintegrate rapidly. The time period for complete disintegration of the dosage unit is typically in the range of from about 10 seconds to about 30 minutes, and optimally is less than 5 minutes.

Other components may also be incorporated into the sublingual dosage forms described herein. The additional components include, but are not limited to binders, disintegrants, wetting agents, lubricants, and the like. Examples of binders that may be used include water, ethanol, polyvinylpyrrolidone, starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual dosage forms are known, or will be apparent, to those skilled in this art; for example, see Remington: The Science and Practice of Pharmacy, cited supra.

Preferred transrectal dosage forms include rectal suppositories, creams, ointments, and liquid formulations (enemas). The suppository, cream, ointment or liquid formulation for transrectal delivery comprises a therapeutically effective amount of the selected androgenic agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration is preferably in the range of from about 10 minutes to about 6 hours, and optimally is less than 3 hours. Other components may also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

The active agents may also be administered intranasally or by inhalation. Compositions for nasal administration are generally liquid formulations for administration as a spray or in the form of drops, although powder formulations for intranasal administration, e.g., insufflations, are also known.

Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, Veegum and combinations thereof). Non-aerosol formulations for inhalation may also comprise dry powder formulations, particularly insufflations in which the powder has an average particle size of about 0.1 μm to 50 μm, preferably 1 μm to about 25 μm.

D. Transdermal Systems:

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdernal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present; the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well know in the art and include, for example, those enhancers listed above in part (B) of this section.

E. Parenteral Formulations:

Parenteral administration, if used, is generally characterized by injection, including intramuscular, intraperitoneal, intravenous (IV) and subcutaneous injection. Injectable formulations can be prepared in conventional forms, either as liquid solutions or suspensions, solid forms suitable for solution or suspension in liquid prior to injection, or as emulsions. Preferably, sterile injectable suspensions are formulated according to techniques known in the art using suitable dispersing or wetting agents and suspending agents. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. A more recently revised approach for parenteral administration involves use of a slow release or sustained release system; see, e.g., U.S. Pat. No. 3,710,795.

IV. Administration:

Preferred dosage forms contain a unit dose of active agent, i.e., a single therapeutically effective dose. For creams, ointments, etc., a “unit dose” requires an active agent concentration that provides a unit dose in a specified quantity of the formulation to be applied, as explained supra. The unit dose of any particular active agent will depend, of course, on the active agent, the needs of the patient, and on the mode of administration. Those of ordinary skill in the art of pharmaceutical formulation can readily deduce suitable unit doses for various androgenic agents, as well as suitable unit doses for other types of active agents that may be incorporated into a dosage form of the invention. For oral administration, the upper end of the dose range is somewhat to substantially higher than the maximum suggested dose for other modes of administration. That is, unit doses in oral dosage forms, for either as-needed or chronic administration, are in the range of about 1 μg (0.001 mg) to about 250 mg, preferably in the range of about 1 μg to about 150 mg, most preferably in the range of about 10 μg to about 100 mg, while unit doses for other types of formulations are in the range of about 1 μg to about 100 mg, preferably about 50 μg to about 50 mg, most preferably about 1 mg to about 25 mg.

Drug administration is on an as-needed basis, and does not involve chronic drug administration. With an immediate release dosage form, i.e., a composition or dosage form that is not “controlled release” as defined hereinabove, as-needed administration may involve drug administration immediately prior to sexual activity, but will generally be in the range of about 0.25 to 72 hours, preferably about 0.5 to 48 hours, more preferably about 1 to 24 hours, most preferably about 1 to 12 hours, and optimally about 1 to 4 hours prior to anticipated sexual activity. As will be appreciated by those in the fields of pharmacology and drug delivery, the upper end of the aforementioned ranges will depend on the pharmacokinetics of the particular active agent administered.

With a sustained release dosage form, a single “as-needed” dose can provide therapeutic efficacy over an extended time period in the range of about 4 to 72 hours, typically in the range of about 4 to 48 hours, more typically in the range of about 4 to 24 hours, depending on the formulation. The release period may be varied by the selection and relative quantity of particular sustained release polymers; see Section III, part (A).

It is not necessary, in either case, to carry out chronic drug administration, i.e., regular dosing on an ongoing basis (such as on a weekly basis, twice weekly, daily, etc.).

As-needed administration as described herein has several advantages over chronic pharmacologic intervention. First, chronic administration of some agents, in particular steroids, can result in serious medical complications and alter the balance of naturally occurring steroids in the body. Second, patient compliance can be problematic with a regimented dosing scheme. Furthermore, as-needed administration is convenient and doses are taken only in anticipation of sexual activity. Thus, needless expenditure on wasted dosages is avoided, thereby decreasing the treatment's overall expense.

The patient treated may be a woman suffering from some type of sexual dysfunction or disorder, or may possess “normal” sexual desire and/or “normal” sexual responsiveness as those terms are understood defined by clinicians or other experts. For these “normal” women, the present invention offers heightened sexual desire and responsiveness relative to her typical sexual experience. Often, however, the female patient seeking enhanced sexual desire and responsiveness suffers a sexual dysfunction such as a condition, disease or disorder that affects one of the four stages of the female sexual response: excitement, plateau, orgasm, or resolution. More specifically, the patient may suffer from any one or more of the following: a decrease in or absence of female sexual responsiveness or female sexual desire; a persistent or recurrent deficiency or absence of sexual fantasies and desire for sexual activity; a decrease in the physiological response to sexual stimulation such as, but not limited to, slowed, decreased or absent erectile response of the female erectile tissues; slowed, decrease or absent lubrication of the vagina; slowed decreased or absent ability to reach orgasm, and decreased intensity of or pleasure in orgasms; frigidity; sexual aversion; a condition, disease or disorder that may result in decreased sexual desire and responsiveness including, but not limited to, the menopausal or post-menopausal state, radiotherapy of the pelvis, multiple sclerosis, atherosclerosis, pelvic trauma or surgery, peripheral neuropathies, autonomic neuropathies, diabetes mellitus, and disorders of the innervation of any of the sexual organs; substance-induced decreases in sexual desire and responsiveness including, but not limited to, decreases related to the administration of pharmacologic agents such as, but not limited to, anti-depressants, neuroleptics, anti-hypertensives, opiates, alcohol, and any other agent found to decrease or eliminate any part of the sexual response cycle; and primary and secondary anorgasmia.

In addition to enhancing female sexual desire and responsiveness, the method, compositions and dosage forms of the invention are useful in improving the tissue health of the female genitalia and preventing vaginal atrophy, preventing pain during intercourse as a result of dyspareunia, and alleviating vaginal itching and dryness associated with dyspareunia and other conditions.

V. Packaged Kits:

In another embodiment, a packaged kit is provided that contains the pharmaceutical formulation to be administered, i.e., a pharmaceutical formulation containing an androgenic agent for enhancing female sexual desire and responsiveness, a container (e.g., a vial, a bottle, a pouch, an envelope, a can, a tube, an atomizer, an aerosol can, etc.), preferably sealed, for housing the formulation during storage and prior to use, and instructions for carrying out drug administration in a manner effective to enhance sexual desire and responsiveness. The instructions will typically be written instructions on a package insert, a label, and/or on other components of the kit.

Depending on the type of formulation and the intended mode of administration, the kit may also include a device for administering the formulation (e.g., a transdermal delivery device). The administration device may be a dropper, a swab, a stick, or the nozzle or outlet of an atomizer or aerosol can. The formulation may be any suitable formulation as described herein. For example, the formulation may be an oral dosage form containing a unit dosage of the androgenic agent, or a gel or ointment contained within a tube. The kit may contain multiple formulations of different dosages of the same agent. The kit may also contain multiple formulations of different active agents.

Examples of Preferred Kits Include:

A kit that includes a container capable of holding 1 to 100 unit doses of the androgenic agent or the pharmaceutical composition containing the androgenic agent, and a dropper that can dispense between 1.0 μg to 50 mg, preferably about 10 μg to 15 mg, of the active agent, as a unit dose. The container is preferably glass, metal, or a plastic known not to adsorb hydrophobic compounds.

B. A kit that includes a container capable of holding 1 to 100 unit doses of the androgenic agent or the pharmaceutical composition containing the androgenic agent, and a spray or aerosol applicator to spray the androgenic agent or pharmaceutical composition, in the form of a liquid or foam, onto the vulvar region of the patient. The container is preferably glass, metal, or a plastic known not to adsorb hydrophobic compounds.

C. A kit that includes a tube capable holding 1 to 100 unit doses of a pharmaceutical composition containing the androgenic agent, which is in the form of a cream or gel, and an applicator that can dispense a unit dose of the composition.

D. A kit that includes 1 to 100 unit doses of the androgenic agent in the form of pellets, a film or suppositories, each individually wrapped in foil or plastic and sealed to protect the active agent and other components of the dosage form from air. The foil or plastic is preferably opaque to eliminate the potentially degrading effects of light on the active agent and other components of the dosage form.

E. A kit that includes 1 to 100 unit doses of a pharmaceutical composition containing the active agent, with the composition having been lyophilized and sealed under inert gas in an ampoule or vial. Lyophilized compositions typically exhibit a much longer shelf life than other dosage forms and may be reconstituted close to the time of use so that the potential for degradation of the active agent is minimized. The kit may also include a suitable diluent, syringe and needle, and/or alcohol swabs.

The present kits will also typically include means for packaging the individual kit components, i.e., the pharmaceutical dosage forms, the administration device (if included), and the written instructions for use. Such packaging means may take the form of a cardboard or paper box, a plastic or foil pouch, etc.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees C. and pressure is at or near atmospheric pressure at sea level. All reagents were obtained commercially unless otherwise indicated.

### Example 1

A cream formulation is prepared using testosterone enanthate. The cream includes the following components:

| | |
|---|---|
| Testosterone enanthate | 100 mg |
| Beeswax | 2.7 gm |
| Carbopol ® 934 q.s. | 100.0 gm |

Mixing is conducted with tile and spatula until a homogeneous cream mixture is obtained having testosterone enanthate uniformly dispersed throughout the formulation.

### Example 2

The procedure of Example 1 is repeated except that the following components are used:

| | |
|---|---|
| Testosterone propionate | 100 mg |
| Polyethylene glycol 400 | 37.5 gm |
| 1,2,6-hexanetriol | 20.0 gm |
| Polyethylene glycol 4000 q.s. | 100.0 gm |

A homogenous cream mixture is obtained.

### Example 3

The procedure of Example 1 is repeated except that the following components are used:

| | |
|---|---|
| Testosterone cypionate | 100 mg |
| Polyethylene glycol 400 | 37.0 gm |
| Polyethylene glycol 400 monostearate | 26.0 gm |
| Polyethylene glycol 4000 q.s. | 100.0 gm |

A homogenous cream mixture is obtained.

### Example 4

The procedure of Example 1 is repeated except that the following components are used:

| | |
|---|---|
| Testosterone enanthate | 100 mg |
| Polyethylene glycol 400 | 47.5 gm |
| Cetyl Alcohol | 5.0 gm |
| Polyethylene glycol 4000 q.s. | 100.0 gm |

A homogenous cream mixture is obtained.

### Example 5

An ointment formulation is prepared using testosterone propionate. The ointment includes the following components:

| | |
|---|---|
| Testosterone propionate | 100 mg |
| Anhydrous lanolin | 20.0 gm |
| Mineral oil | 25.0 gm |
| White Petrolatum q.s. | 100.0 gm |

Mixing is conducted with tile and spatula until a homogeneous ointment mixture is obtained having the testosterone propionate uniformly dispersed throughout the formulation.

### Example 6

The procedure of Example 5 is repeated except that the following components are used:

| | |
|---|---|
| Testosterone cypionate | 100 mg |
| Diisopropyl Adipate | 19.95 gm |
| White Petrolatum, USP q.s. | 100.0 gm |

A homogenous ointment mixture is obtained.

In the cream and ointment formulations described in Examples 1-6, optional ingredients can include materials such as antioxidants, viscosity modifiers (e.g., paraffin wax or lanolin wax), and topical absorption rate modifiers.

### Example 7

A vaginal suppository formulation is prepared that contains testosterone cypionate. The suppository includes the following components:

| | |
|---|---|
| Testosterone cypionate | 100 mg |
| Polyethylene glycol 400 | 37.0 gm |
| Glycerol gelatin | 20.0 gm |
| Polyethylene glycol 4000 q.s. | 100.0 gm |

The polyethylene glycol 400 solution containing testosterone cypionate is mixed well with glycerol gelatin or similar suppository base such as macrogol, Witepsol®, or the like, with a mechanical mixing apparatus, and the mixture is then cooled in a suppository mold.

### Example 8

Suppositories suitable for vaginal administration of a unit dosage of testosterone propionate are prepared. A pharmaceutical formulation containing testosterone propionate is prepared by mixing the androgen with polyethylene glycol, molecular weight (M_{w}) approximately 4000, and heating the mixture to a temperature just high enough to produce a prostaglandin-polymer melt. The androgen-glycol mixture can then be poured into a mold suitable to provide a suppository, and allowed to cool. The suppository so provided is a unit dosage form suitable for transurethral administration. If desired, the androgen-glycol mixture may be allowed to cool on the tip of a rod adapted to be inserted into the urethra.

### Example 9

Individuals are assessed and pre-screened to assemble an experimental group of female subjects seeking enhanced sexual desire and responsiveness. The formulations prepared in Examples 1-6 are assessed in the experimental subjects for their ability to increase uterine or vaginal epithelial blood flow. One gram of formulation is applied topically to the clitoris and within the vulvar region to provide a dose of about 1 mg androgenic agent, and changes in blood flow or vaginal fluid production four hours after application of the formulations are determined using known methods. Increase in vaginal epithelial blood flow is determined using indirect methods such as photoplethysmography (Levin (1980) Clinics in Obstet. Gynaecol. 7:213-252), heated oxygen electrode (Wagner et al. (1978), “Vaginal Fluid” in The Human Vagina, Evans et al. (eds.), Amsterdam: Elsevier/North Holland Biomedical Press, pp. 121-137), and direct clearance of radioactive Xenon (Wagner et al. (1980) Obstet. Gynaecol. 56:621-624). Changes in vulvar blood flow are monitored using laser Doppler velocimetry (Sarrel, P. M. (1990) Obstet. Gynaecol. 75:26S-32S).

Decreased vaginal dryness and/or dyspareunia are negatively correlated with vaginal blood flow rates, wherein increased blood flow to the vagina correlates with increased lubrication and decreased frequency and severity of dyspareunia (Sarrel, P. M. (1990) Obstet. Gynaecol. 75:26S-32S). Accordingly, vulvar blood flow after treatment is assessed using laser Doppler velocimetry and compared to baseline levels. Increased vaginal lubrication as a result of treatment with the formulations can also be assessed using the methods of Semmens et al. (1982) J. Am. Med. Assoc. 248:445-448. The formulations of Examples 1-6, when assessed using such methods, are found to substantially increase blood flow to the vagina and vulvar area and alleviate vaginal dryness.

### Example 10

The method of Example 9 is repeated using the suppository formulations of Examples 8 and 9. Substantially the same results are obtained.

### Example 11

The method of Example 9 is repeated, but the active agent is administered orally in the form of a tablet containing 100 μg of testosterone ethyl ester, i.e., testosterone propionate. Substantially the same results are obtained.

### Example 12

The method of Example 11 is repeated, but a topical formulation containing 500 μg prostaglandin E₁ is administered one hour after administration of the testosterone ethyl ester. Relative to Examples 10 and 11, this method is found to even more significantly increase blood flow to the vagina and vulvar area and alleviate vaginal dryness.

1. A method for enhancing sexual desire and responsiveness in a female individual, comprising administering to the individual a therapeutically effective amount of an androgenic agent on an as-needed basis without regular dosing within the context of a chronic dosage regimen.

2. The method of claim 1, wherein the androgenic agent is contained within a pharmaceutical formulation.

3. The method of claim 2, wherein the pharmaceutical formulation is comprised of an immediate release dosage form, and the androgenic agent is administered about 0.25 to 72 hours prior to anticipated sexual activity.

4. The method of claim 3, wherein the androgenic agent is administered about 0.5 to 48 hours prior to anticipated sexual activity.

5. The method of claim 4, wherein the androgenic agent is administered about 1 to 24 hours prior to anticipated sexual activity.

6. The method of claim 5, wherein the androgenic agent is administered about 1 to 12 hours prior to anticipated sexual activity.

7. The method of claim 6, wherein the androgenic agent is administered about 1 to 4 hours prior to anticipated sexual activity.

8. The method of claim 2, wherein the pharmaceutical formulation is comprised of a sustained release dosage form.

9. The method of claim 8, wherein following administration, the sustained release dosage form provides release of the androgenic agent over a drug delivery period in the range of about 4 to 72 hours.

10. The method of claim 9, wherein the drug delivery period is in the range of about 4 to 48 hours.

11. The method of claim 10, wherein the drug delivery period is in the range of about 4 to 24 hours.

12. The method of claim 2 wherein the androgenic agent is selected from the group consisting of androsterone, androstenediol, androstenedione, ethylestrenol, oxandrol one, nandrolone, stanozolol, dromostanolone, testosterone, dehydroepiandrosterone, 4-dihydrotestosterone, methyl testosterone, testolactone, oxymetholone, fluoxymesterone, pharmacologically active salts and esters thereof, and combinations of any of the foregoing.

13. The method of claim 12, wherein the androgenic agent is selected from the group consisting of testosterone, 4-dihydrotestosterone, and pharmacologically active esters thereof.

14. The method of claim 13, wherein the androgenic agent is selected from the group consisting of testosterone and pharmacologically active esters thereof.

15. The method of claim 14, wherein the androgenic agent is testosterone.

16. The method of claim 14, wherein the androgenic agent is a pharmacologically active testosterone ester.

17. The method of claim 15, wherein the testosterone ester is selected from the group consisting of testosterone enanthate, propionate, cypionate, phenylacetate, acetate, buciclate, heptanoate, decanoate, undecanoate, caprate, isocaprate, and C₄-C₆ alkyl-substituted cycloalkylcarboxylates.

18. The method of claim 17, wherein the testosterone ester is testosterone propionate, testosterone undecanoate, testosterone C₄-C₆ alkyl-substituted cyclobutanecarboxylate, testosterone C₄-C₆ alkyl-substituted cyclopentanecarboxylates, and testosterone C₄-C₆ alkyl-substituted cyclohexanecarboxylates.

19. The method of claim 12, wherein the androgenic agent is dehydroepiandrosterone.

20. The method of claim 12, wherein the therapeutically effective amount is in the range of about 1 μg to about 250 mg.

21. The method of claim 20, wherein the therapeutically effective amount is in the range of about 1 μg to about 150 mg.

22. The method of claim 21, wherein the therapeutically effective amount is in the range of about 10 μg to about 100 mg.

23. The method of claim 2, wherein the pharmaceutical formulation is administered to the patient's vulvar region and/or vagina.

24. The method of claim 23, wherein the therapeutically effective amount is in the range of about 1 μg to about 100 mg.

25. The method of claim 24, wherein the therapeutically effective amount is in the range of about 50 μg to about 50 mg.

26. The method of claim 25, wherein the therapeutically effective amount is in the range of about 1.0 mg to 25 mg.

27. The method of claim 23, wherein the pharmaceutical formulation is a topical formulation, and is administered to the patient's vulvar region.

28. The method of claim 23, wherein the pharmaceutical formulation is suitable for vaginal administration and is administered vaginally.

29. The method of claim 1, wherein administration is transdermal, sublingual, intranasal, buccal, rectal, parenteral, or by inhalation.

30. The method of claim 2, wherein the pharmaceutical formulation comprises a unit dosage form.

31. The method of claim 1, further comprising administering a therapeutically effective amount of at least one additional active agent.

32. The method of claim 31, wherein the at least one additional active agent is administered with the androgenic agent.

33. The method of claim 31, wherein the at least one additional active agent is administered prior to administration of the androgenic agent.

34. The method of claim 31, wherein the at least one additional active agent is administered after administration of the androgenic agent.

35. The method of claim 31, wherein the at least one additional active agent is a vasoactive agent.

36. The method of claim 35, wherein the vasoactive agent is a vasodilator.

37. The method of claim 36, wherein the vasodilator is selected from the group consisting of vasoactive prostaglandins, endothelin-derived relaxation factors, vasoactive intestinal polypeptide agonists, smooth muscle relaxants, leukotriene inhibitors, and pharmacologically active salts, esters, prodrugs, and metabolites thereof, and combinations of any of the foregoing.

38. The method of claim 37, wherein the vasodilator is a vasoactive prostaglandin.

39. The method of claim 37, wherein the vasoactive prostaglandin is selected from the group consisting of naturally occurring prostaglandins, semisynthetic prostaglandins, synthetic prostaglandins, and pharmaceutically acceptable, pharmacologically active salts, esters, amides, inclusion complexes, prodrugs, metabolites, and analogs thereof, and combinations of any of the foregoing.

40. The method of claim 39, wherein the vasoactive prostaglandin is selected from the group consisting of naturally occurring prostaglandins and hydrolyzable lower alkyl esters thereof.

41. The method of claim 40, wherein the vasoactive prostaglandin is selected from the group consisting of PGE₀, PGE₁, 19-hydroxy-PGE₁, PGE₂, 19-hydroxy-PGE₂, PGA₁, 19-hydroxy-PGA₁, PGA₂, 19-hydroxy-PGA₂, PGB₁, 19-hydroxy-PGB₁, PGB₂, 19-hydroxy-PGB₂, PGB₃, PGD₂, PGF_{1α}, PGF_{2α}, PGE₃, PGF_{3α}, PGI₂, and hydrolyzable lower alkyl esters thereof.

42. The method of claim 41, wherein the vasoactive prostaglandin is selected from the group consisting of PGE₀, PGE₁, PGE₂, and the methyl, ethyl and isopropyl esters thereof.

43. The method of claim 38, wherein the vasoactive prostaglandin is selected from the group consisting of arboprostil, carbaprostacyclin, carboprost tromethamine, dinoprost tromethamine, dinoprostone, enprostil, iloprost, lipoprost, gemeprost, metenoprost, sulprostone, tiaprost, viprostil, viprostil methyl ester, 16,16-dimethyl-Δ²-PGE, methyl ester, 15-deoxy-16-hydroxy-16-methyl-PGE₁ methyl ester, 16,16-dimethyl-PGE₁, 11-deoxy-15-methyl-PGE ₁, 16-methyl-18,18,19,19-tetrahydro-carbacyclin, 16(RS)-15-deoxy-16-hydroxy-16-methyl-PGE₁ methyl ester, (+)-4,5-didehydro-16-phenoxy-α-tetranor-PGE₂ methyl ester, 11-deoxy-11α,16,16-trimethyl-PGE₂, (+)-11α,16α,16β-dihydroxy-1,9-dioxo-1-(hydroxymethyl)-16-methyl-transprostene, 9-chloro-16,16-dimethyl-PGE₂, 16,16-dimethyl-PGE₂, 15(S)-15-methyl-PGE₂, 9-deoxy-9-methylene-16,16-dimethyl-PGE₂, potassium salt, 19(R)-hydroxy-PGE₂, 11-deoxy-16,16-dimethyl-PGE₂, and combinations thereof.

44. The method of claim 38, wherein the therapeutically effective amount of the vasodilator is in the range of approximately 1 to 5000 μg.

45. The method of claim 44, wherein the therapeutically effective amount of the vasodilator is in the range of approximately 20 to 2000 μg.

46. The method of claim 31, wherein the additional active agent is selected from the group consisting of rho kinase inhibitors, melanocortin peptides, endothelin antagonists, growth factors and other peptidyl drugs; selective androgen receptor modulators (SARMs), neuropeptides, amino acids, serotonin agonists, serotonin antagonists, calcium channel blockers, potassium channel openers, potassium channel blockers, dopamine agonists, dopamine antagonists, non-androgenic steroids, and combinations thereof.

47. The method of claim 46, wherein the additional active agent is a dopamine agonist.

48. The method of claim 47, wherein the dopamine agonist is selected from the group consisting of levodopa, bromocriptine, pergolide, apomorphine, piribedil, pramipexole, ropinirole, and combinations thereof.

49. A method for enhancing sexual desire and responsiveness in a female individual, comprising administering to the individual, approximately 0.25 to 72 hours prior to anticipated sexual activity, a therapeutically effective amount of an androgenic agent, followed by administration, approximately 0.25 to 24 hours prior to anticipated sexual activity, of a therapeutically effective amount of a prostaglandin.

50. A method for maintaining improving the tissue health of the female genitalia, comprising administering to a female individual, on an as-needed basis, a therapeutically effective amount of an androgenic agent.

51. A method for preventing vaginal atrophy, comprising administering to a female individual, on an as-needed basis, a therapeutically effective amount of an androgenic agent.

52. A method for preventing vaginal pain during sexual intercourse, comprising administering to a female individual suffering from dyspareunia a therapeutically effective amount of an androgenic agent, on an as-needed basis.

53. A method for alleviating vaginal itching and dryness, comprising administering to a female individual in need of such treatment a therapeutically effective amount of an androgenic agent, on an as-needed basis.

54. A method for enhancing sexual desire and responsiveness in a female individual, comprising administering an androgenic agent to the individual in an amount effective to provide a blood level of the agent or a metabolite thereof that approximates the blood level of the agent or a metabolite thereof during ovulation, wherein said administering is on an as-needed basis without regular dosing within the context of a chronic dosage regimen.

55. A pharmaceutical formulation for enhancing female sexual desire and responsiveness, comprising (a) approximately 1.0 μg to 500 mg androgenic agent per gram of formulation, (b) a pharmaceutically acceptable carrier suitable for vaginal and/or vulvar administration and selected to provide immediate release of the androgenic agent from the formulation following application to the individual's vagina and/or vulvar area, such that the formulation may be effectively administered on an on-demand basis.

56. The formulation of claim 58 wherein the androgenic agent is selected from the group consisting of androsterone, androstenediol, androstenedione, ethylestrenol, oxandrolone, nandrolone phenpropionate, nandrolone, stanozolol, dromostanolone, testosterone, dehydroepiandrosterone, 4-dihydrotestosterone, methyl testosterone, testolactone, oxymetholone, fluoxymesterone, pharmacologically active salts and esters thereof, and combinations of any of the foregoing.

57. The formulation of claim 56, wherein the androgenic agent is selected from the group consisting of testosterone, 4-dihydrotestosterone, and pharmacologically active esters thereof.

58. The formulation of claim 57, wherein the androgenic agent is selected from the group consisting of testosterone and pharmacologically active esters thereof.

59. The formulation of claim 58, wherein the androgenic agent is testosterone.

60. The formulation of claim 58, wherein the androgenic agent is a pharmacologically active testosterone ester.

61. The formulation of claim 58, containing approximately 1.0 μg to 150 mg androgenic agent per gram of formulation

62. A packaged kit for a female individual to use in enhancing sexual desire and responsiveness, comprising: a pharmaceutical formulation of an androgenic agent; a container housing the pharmaceutical formulation during storage and prior to administration; and instructions for carrying out drug administration to enhance sexual desire and responsiveness, on an as-needed basis.
